(19) United States
(12) Patent Application Publication
(10) Pub. No.: US 2026/0211110 A1
(43) Pub. Date: Jul. 23, 2026

(54) ULTRASOUND IMAGING DEVICE AND ULTRASOUND IMAGING METHOD

(71) Applicant: SHENZHEN MINDRAY BIO-MEDICAL ELECTRONICS CO., LTD., Shenzhen (CN)

(72) Inventors: Zhiye HUANG, Shenzhen (CN); Wei FAN, Shenzhen (CN); Lei ZHANG, Shenzhen (CN); Zhuangzhi YU, Shenzhen (CN)

(73) Assignee: SHENZHEN MINDRAY BIO-MEDICAL ELECTRONICS CO., LTD., Shenzhen (CN)

(21) Appl. No.: 19/455,097

(22) Filed: Jan. 21, 2026

(30) Foreign Application Priority Data
Jan. 22, 2025 (CN) 202510112773.7

(51) Int. Cl. G01S 15/89; G01S 7/52

(52) U.S. Cl. CPC G01S 15/8927; G01S 7/52074; G01S 15/8925

(57) ABSTRACT

An ultrasound imaging device and method are disclosed, where, multiple transmitting apertures are provided on an ultrasound probe, and an ultrasound beam is transmitted along at least one corresponding scanning line by one transmitting aperture, thereby defining multiple scanning planes. Since the multiple scanning lines in the same scanning plane intersect at an endpoint in the opposite direction of the transmitting of the ultrasound beam, and the endpoint and the area being scanned are located on both sides of the array element, the imaging range in space is larger than that of conventional sector scanning, thereby maximizing the near-field field of view.

### CROSS-REFERENCE TO RELATED APPLICATION

The present application claims priority to Chinese Patent Application No. 202510112773.7 filed on January 22, 2025, the disclosure of which is hereby incorporated by reference in its entirety.

### TECHNICAL FIELD

The present disclosure relates to ultrasound imaging, specifically to an ultrasound imaging device and an ultrasound imaging method.

### BACKGROUND

Intracardiac echocardiography (ICE) is an ultrasound imaging technique in which the catheter ultrasound probe is inserted into the heart chamber through the peripheral blood vessels to provide real-time, high-quality imaging and/or hemodynamic measurements to the heart and the adjacent tissues thereof. Because the catheter ultrasound probe enters the heart chambers, the imaging depth is generally shallow (6-8 cm), and the requirement to visualization of the cardiac structures is high. Currently, conventional sector scanning is commonly used, which has small near-field field of view. Therefore, some near-field structural information will be lost in the final 2D ultrasound images, which causes the possibility of missed diagnosis or misdiagnosis.

### SUMMARY

In an embodiment, an ultrasound imaging device is provided, which may include: an ultrasound probe, where, the ultrasound probe includes an array element group comprising multiple array elements, the array element group forms multiple transmitting aperture groups, each transmitting aperture group includes at least two transmitting apertures, each transmitting aperture includes multiple array elements, and each transmitting aperture transmits ultrasound beams along at least one scan line corresponding to said transmitting aperture; and

### a processor configured to:

control the multiple transmitting aperture groups formed by the array element group to respectively transmit ultrasound beams toward an area being scanned, where:

for any one of the multiple transmitting aperture groups, the ultrasound beam is transmitted to the area being scanned by each transmitting aperture in the transmitting aperture group along at least one scan line corresponding to said transmitting aperture, and the multiple scan lines corresponding to the multiple transmitting apertures in the transmitting aperture group determine one scanning plane corresponding to said transmitting aperture group in space, where, each scanning plane includes multiple scanning lines, the multiple scanning lines in a same scanning plane intersect at an endpoint in a direction opposite to a transmitting direction of the ultrasound beams, each scanning plane has a corresponding endpoint, and the endpoint is located on a side of the of the array element group opposite to the area being scanned;

sequentially receive ultrasound echoes of the ultrasound beam transmitted by each transmitting aperture group reflected from the area being scanned via the array element group to obtain echo signals, sequentially generate multiple 2D ultrasound images of the area being scanned according to the echo signals corresponding to each transmitting aperture group, and display the multiple 2D ultrasound images.

In an embodiment, the array element group includes N rows and M columns of transmitting apertures, where

at least two transmitting apertures in each row form one transmitting aperture group; and/or, at least two transmitting apertures in each column form one transmitting aperture group.

In an embodiment, the multiple scanning planes corresponding to the multiple transmitting aperture groups include a first scanning plane and a second scanning plane, and the endpoint corresponding to the first scanning plane coincide with the endpoint corresponding to the second scanning plane.

In an embodiment, an arrangement direction of transmitting array elements corresponding to the multiple scan lines in the first scanning plane is parallel to an arrangement direction of transmitting array elements corresponding to the multiple scan lines in the second scanning plane.

In an embodiment, an arrangement direction of transmitting array elements corresponding to the multiple scan lines in the first scanning plane is perpendicular to an arrangement direction of transmitting array elements corresponding to the multiple scan lines in the second scanning plane.

In an embodiment, the scanning planes corresponding to the multiple transmitting apertures include a third scanning plane and a fourth scanning plane, and the endpoint corresponding to the third scanning plane, the endpoint corresponding to the fourth scanning plane and a center of the array element group are located on a same straight line.

In an embodiment, the endpoint corresponding to the third scanning plane and the endpoint corresponding to the fourth scanning plane are located on the straight line passing through the center of the array element group in a vertical direction.

In an embodiment, an arrangement direction of transmitting array elements corresponding to the multiple scanning lines in the third scanning plane is parallel to an arrangement direction of transmitting array elements corresponding to the multiple scanning lines in the fourth scanning plane.

In an embodiment, an arrangement direction of transmitting array elements corresponding to the multiple scanning lines in the third scanning plane is perpendicular to an arrangement direction of transmitting array elements corresponding to the multiple scanning lines in the fourth scanning plane.

In an embodiment, a projection of the array element group in a vertical direction is a rectangle, and the scanning plane is configured as:

a projection line of the scanning plane in a vertical direction passes through a center of the rectangle.

In an embodiment, an ultrasound imaging device is provided, which may include:

an ultrasound probe, where, the ultrasound probe includes an array element group comprising multiple array elements, the array element group forms multiple transmitting aperture groups, each transmitting aperture group includes at least two transmitting apertures, each transmitting aperture includes multiple array elements, the multiple transmitting apertures are arranged along a first direction, and one transmitting aperture transmits ultrasound beams along at least one scan line corresponding to said transmitting aperture; and

### a processor configured to:

control the multiple transmitting apertures formed by the array element group to respectively transmit ultrasound beams to an area being scanned, where

for any one of the multiple transmitting aperture groups, the ultrasound beams are transmitted to the area being scanned by each transmitting aperture in the transmitting aperture group along at least one scan line corresponding to said transmitting aperture, where the multiple scan lines corresponding to the multiple transmitting apertures in said transmitting aperture group determine a scanning plane corresponding to said transmitting aperture group in space;

for the scanning planes corresponding to any two transmitting aperture groups in the multiple transmitting aperture groups, a centerline of the scanning plane corresponding to one transmitting aperture group and a centerline of the scanning plane corresponding to the other transmitting aperture group intersect at an endpoint in a direction opposite to a transmitting direction of the ultrasound beams and the endpoint is located on a side of the area being scanned opposite to the array element group;

sequentially receive ultrasound echoes of the ultrasound beam transmitted by each transmitting aperture group reflected from the area being scanned via the array element group to obtain echo signals, sequentially generate multiple 2D ultrasound images of the area being scanned according to the echo signals corresponding to each transmitting aperture group, and display the multiple 2D ultrasound images.

In an embodiment, the scanning planes corresponding to the transmitting aperture groups are parallel to a second direction, and the second direction is perpendicular to the first direction.

In an embodiment, an ultrasound imaging device is provided, which may include:

an ultrasound probe comprising an array element group, where the array element group includes multiple array elements; and

### a processor configured to:

control multiple transmitting aperture groups formed by the array element group to respectively transmit ultrasound beams to an area being scanned, where

for any one transmitting aperture group in the multiple transmitting aperture groups, the ultrasound beams are transmitted to the area being scanned by each transmitting aperture in said transmitting aperture group along at least one scan line corresponding to said transmitting aperture, the multiple scan lines corresponding to the multiple transmitting apertures in said transmitting aperture group determine one scanning plane corresponding to said transmitting aperture group in space, each scanning plane includes multiple scan lines, the multiple scan lines in a same scanning plane intersect at an endpoint in a direction opposite to a transmitting direction of the ultrasound beam, each scanning plane has a corresponding endpoint, the endpoint is located on a side of the array element group opposite to the area being scanned, and the scanning planes corresponding to the multiple transmitting aperture groups intersect the array element group at a same straight line located on the array element group;

sequentially receive ultrasound echoes of the ultrasound beam transmitted by each transmitting aperture group reflected from the area being scanned via the array element group to obtain echo signals, sequentially generate multiple 2D ultrasound images of the area being scanned according to the echo signals corresponding to each transmitting aperture group, and display the multiple 2D ultrasound images.

In an embodiment, an ultrasound imaging method is provided, which may be applied to an ultrasound imaging device. The ultrasound imaging device includes an ultrasound probe comprising an array element group. The array element group includes multiple array elements. The array element group forms multiple transmitting aperture groups, each transmitting aperture group includes at least two transmitting apertures, and each transmitting aperture includes several array elements. One transmitting aperture transmits ultrasound beams along at least one scanning line corresponding to said one transmitting aperture. The method includes:

control the multiple transmitting aperture groups formed by the array element group to respectively transmit the ultrasound beams to an area being scanned, where

for any one of the multiple transmitting aperture groups, the ultrasound beam is transmitted toward the area being scanned by each transmitting aperture in the transmitting aperture group along at least one scan line corresponding to said transmitting aperture, and the multiple scan lines corresponding to the multiple transmitting apertures in the transmitting aperture group determine one scanning plane corresponding to said transmitting aperture group in space, where, each scanning plane includes multiple scanning lines, the multiple scanning lines in a same scanning plane intersect at an endpoint in a direction opposite to a transmitting direction of the ultrasound beams, each scanning plane has a corresponding endpoint, and the endpoint is located on a side of the of the array element group opposite to the area being scanned;

sequentially receive ultrasound echoes of the ultrasound beam transmitted by each transmitting aperture group reflected from the area being scanned via the array element group to obtain echo signals, sequentially generate multiple 2D ultrasound images of the area being scanned according to the echo signals corresponding to each transmitting aperture group, and display the multiple 2D ultrasound images.

In an embodiment, the array element group includes N rows and M columns of transmitting apertures, where

at least two transmitting apertures in each row form one transmitting aperture group;

and/or, at least two transmitting apertures in each column form one transmitting aperture group.

In an embodiment, the scanning planes corresponding to the multiple transmitting aperture groups includes a first scanning plane and a second scanning plane, and the endpoint corresponding to the first scanning plane coincide with the endpoint corresponding to the second scanning plane.

In an embodiment, an arrangement direction of transmitting array elements corresponding to the multiple scan lines in the first scanning plane is parallel to an arrangement direction of transmitting array elements corresponding to the multiple scan lines in the second scanning plane.

In an embodiment, an arrangement direction of transmitting array elements corresponding to the multiple scan lines in the first scanning plane is perpendicular to an arrangement direction of transmitting array elements corresponding to the multiple scan lines in the second scanning plane.

In an embodiment, the scanning planes corresponding to the multiple transmitting aperture groups includes a third scanning plane and a fourth scanning plane, and the endpoint corresponding to the third scanning plane, the endpoint corresponding to the fourth scanning plane and a center of the array element group are located on a same straight line.

In an embodiment, the endpoint corresponding to the third scanning plane and the endpoint corresponding to the fourth scanning plane are located on the straight line passing through the center of the array element group in a vertical direction.

In an embodiment, an arrangement direction of transmitting array elements corresponding to the multiple scanning lines in the third scanning plane is parallel to an arrangement direction of transmitting array elements corresponding to the multiple scanning lines in the fourth scanning plane.

In an embodiment, an arrangement direction of transmitting array elements corresponding to the multiple scanning lines in the third scanning plane is perpendicular to an arrangement direction of transmitting array elements corresponding to the multiple scanning lines in the fourth scanning plane.

In an embodiment, a projection of the array element group in a vertical direction is a rectangle, and the scanning plane is configured as:

a projection line of the scanning plane in a vertical direction passes through a center of the rectangle.

In an embodiment, an ultrasound imaging method is provided, which may be applied to an ultrasound imaging device. The ultrasound imaging device includes an ultrasound probe comprising an array element group. The array element group includes multiple array elements. The array element group forms multiple transmitting aperture groups. Each transmitting aperture group includes at least two transmitting apertures, and each transmitting aperture includes several array elements. Multiple transmitting apertures are arranged along a first direction. One transmitting aperture transmits ultrasound beams along at least one scanning line corresponding to said one transmitting aperture. The method includes:

control the multiple transmitting aperture groups formed by the array element group to respectively transmit the ultrasound beams to an area being scanned, where

for any one of the multiple transmitting aperture groups, the ultrasound beam is transmitted toward the area being scanned by each transmitting aperture in the transmitting aperture group along at least one scan line corresponding to said transmitting aperture, and the multiple scan lines corresponding to the multiple transmitting apertures in the transmitting aperture group determine one scanning plane corresponding to said transmitting aperture group in space;

for the scanning planes corresponding to any two transmitting aperture groups in the multiple transmitting aperture groups, a centerline of the scanning plane corresponding to one transmitting aperture group and a centerline of the scanning plane corresponding to the other transmitting aperture group intersect at an endpoint in a direction opposite to a transmitting direction of the ultrasound beams and the endpoint is located on a side of the area being scanned opposite to the array element group;

sequentially receive ultrasound echoes of the ultrasound beam transmitted by each transmitting aperture group reflected from the area being scanned via the array element group to obtain echo signals, sequentially generate multiple 2D ultrasound images of the area being scanned according to the echo signals corresponding to each transmitting aperture group, and display the multiple 2D ultrasound images.

In an embodiment, the scanning planes are parallel to a second direction that is perpendicular to the first direction.

In an embodiment, an ultrasound imaging method is provided, which may be applied to an ultrasound imaging device. The ultrasound imaging device includes an ultrasound probe comprising an array element group. The array element group includes multiple array elements. The array element group forms multiple transmitting aperture groups, each transmitting aperture group includes at least two transmitting apertures, and each transmitting aperture includes several array elements. One transmitting aperture transmits ultrasound beams along at least one scanning line corresponding to said one transmitting aperture. The method includes:

control the multiple transmitting aperture groups formed by the array element group to respectively transmit the ultrasound beams to an area being scanned, where

for any one of the multiple transmitting aperture groups, the ultrasound beam is transmitted toward the area being scanned by each transmitting aperture in the transmitting aperture group along at least one scan line corresponding to said transmitting aperture, and the multiple scan lines corresponding to the multiple transmitting apertures in the transmitting aperture group determine one scanning plane corresponding to said transmitting aperture group in space, where, each scanning plane includes multiple scanning lines, the multiple scanning lines in a same scanning plane intersect at an endpoint in a direction opposite to a transmitting direction of the ultrasound beams, each scanning plane has a corresponding endpoint, the endpoint is located on a side of the of the array element group opposite to the area being scanned, and the scanning planes corresponding to the multiple transmitting aperture groups and the array element group intersect at a same straight line located on the array element group; and

sequentially receive ultrasound echoes of the ultrasound beam transmitted by each transmitting aperture group reflected from the area being scanned via the array element group to obtain echo signals, sequentially generate multiple 2D ultrasound images of the area being scanned according to the echo signals corresponding to each transmitting aperture group, and display the multiple 2D ultrasound images.

In an embodiment, a computer-readable storage medium storing a program is provided. The program is executed by a processor to implement the method of any one of the embodiments above.

According to the ultrasound imaging devices and methods in the embodiments above, multiple transmitting aperture groups are arranged in the ultrasound probe. For any one of the multiple transmitting aperture groups, the ultrasound beams are transmitted to the area being scanned along at least one corresponding scan line by each transmitting aperture in the transmitting aperture group. The echo signals of the ultrasound beams transmitted by each transmitting aperture group reflected by the area being scanned are received sequentially by the array element group. Multiple 2D ultrasound images of the area being scanned are generated and displayed sequentially according to the echo signals corresponding to each transmitting aperture. The multiple scan lines corresponding to the multiple transmitting apertures in the transmitting aperture group determine a scanning plane corresponding to the transmitting aperture group in space. Since the multiple scan lines in the same scanning plane intersect at an endpoint in the direction opposite to the transmitting direction of the ultrasonic beams, and the endpoint is located on the side of the array element group opposite to the area being scanned, the imaging range in space is larger than that of the conventional sector scanning method, thereby maximizing the near field of view.

### BRIEF DESCRIPTION OF THE DRAWINGS

FIG. 1 is a schematic diagram showing the scanning plane corresponding to the conventional sector scanning;

FIG. 2 is a schematic diagram showing the scanning plane corresponding to the conventional sector scanning and the scanning plane corresponding to the scanning method provided by an embodiment of the present disclosure;

FIG. 3 is a schematic diagram showing the three-dimensional scanning volumes corresponding to the conventional sector scanning and the scanning method provided by an embodiment of the present disclosure;

FIG. 4 is a schematic diagram showing an ultrasound imaging device according to an embodiment of the present disclosure;

FIG. 5 is a flowchart of an ultrasound imaging method according to an embodiment of the present disclosure;

FIG. 6 is a schematic diagram showing the scanning plane corresponding to a transmitting aperture according to an embodiment of the present disclosure;

FIG. 7 is a schematic diagram showing the arrangement of N rows of transmitting apertures according to an embodiment of the present disclosure;

FIG. 8 is a schematic diagram showing the arrangement of M columns of transmitting apertures according to an embodiment of the present disclosure;

FIG. 9 is a schematic diagram showing the arrangement of N rows and M columns of transmitting apertures according to an embodiment of the present disclosure;

FIG. 10 is a schematic diagram showing a three-dimensional scanning volume including M+N scanning planes according to an embodiment of the present disclosure;

FIG. 11 is a schematic diagram showing any two scanning planes corresponding to the transmitting aperture according to an embodiment of the present disclosure;

FIG. 12 is a schematic diagram showing any two scanning planes corresponding to the transmitting aperture according to an embodiment of the present disclosure;

FIG. 13 is a schematic diagram showing the projection lines of the scanning plane corresponding to the scanning method according to an embodiment of the present disclosure; and

FIG. 14 is a schematic diagram showing the projection lines of the scanning plane corresponding to the scanning method according to another embodiment of the present disclosure.

### DETAILED DESCRIPTION

The present disclosure will be described in details below with reference to specific

embodiments and drawings, where similar elements in different embodiments are assigned with similar reference numbers. In the following embodiments, many details are described to facilitate a better understanding of this disclosure. However, those skilled in the art will readily recognize that some features may be omitted in different situations, or may be replaced by other elements, materials, or methods. In some cases, certain operations are not shown or described in the specification, which is to avoid obscuring the core parts of the present disclosure with excessive description. For those skilled in the art, detailed description of these operations is not necessary. They can fully understand the operations based on the description in the specification and general technical knowledge in the art.

Furthermore, the features, operations or characteristics described in the specification can be combined in any suitable manner to form various embodiments. In addition, the described steps or actions of the method can be rearranged or adjusted in a manner obvious to those skilled in the art. Therefore, the various orders in the specification and drawings are merely for the clear description of a particular embodiment, but do not imply a necessary order, unless otherwise stated that a particular order must be followed.

The serial numbers assigned to components in the present disclosure, such as "first" and "second," are used merely to distinguish the described objects, but have no sequential or technical meaning. The terms "connection" and "linkage" used in the present disclosure, unless otherwise specified, include both direct and indirect connections (linkages).

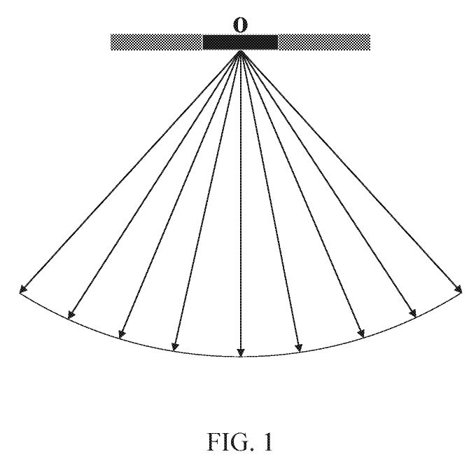

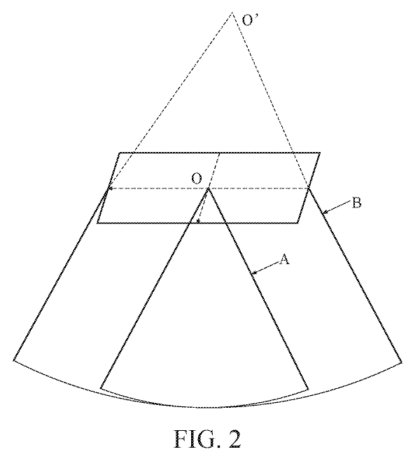

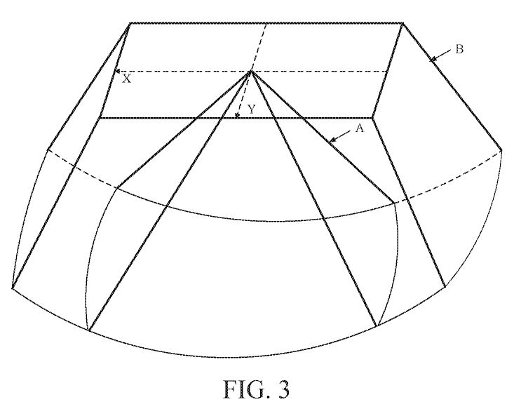

In the conventional sector scanning of an ultrasound probe, a single transmitting aperture formed by one or more array elements of the ultrasound probe transmits ultrasound beams along multiple scan lines toward the area being scanned. The multiple scan lines corresponding to one transmitting aperture may determine a scanning plane, as shown in FIG. 1. The multiple scan lines defining a scanning plane intersect at the transmitting aperture O on the ultrasound probe. In the present disclosure, multiple transmitting apertures formed by the array elements of the ultrasound probe may be provided to transmit the ultrasound beams toward the area being scanned. Each transmitting aperture transmits the ultrasound beam along at least one scan line. The multiple scan lines corresponding to the multiple transmitting apertures can determine multiple scanning planes in space. The multiple scan lines within the same scanning plane intersect at a virtual endpoint in the opposite direction of the transmitting direction of the ultrasound beam. This virtual endpoint and the area being scanned are located on opposite sides of the array elements of the ultrasound probe. Therefore, compared to the conventional sector scanning, the scanning plane of the scanning method of the present disclosure has a larger imaging range, which expands the near-field field of view. For a more intuitive comparison, please refer to FIG. 2. A is a scanning plane corresponding to the conventional sector scanning method, where the scanning lines intersect at point O on the ultrasound probe. B is a scanning plane corresponding to the scanning method provided by the present disclosure, where the scanning lines intersect at point O' on one side of the ultrasound probe. It is clear that in the same scanning angle range, the scanning plane of the scanning method provided by the present disclosure has a larger imaging range than the scanning plane of the conventional sector scanning method, thus providing a larger near-field field of view. Furthermore, please refer to FIG. 3, in the case that the ultrasound scanning is performed in the X and Y directions of the ultrasound probe, the scanning method (B) provided by the present disclosure also has a larger near-field field of view compared to the conventional sector scanning method (A). The following detailed descriptions will be provided through some specific embodiments.

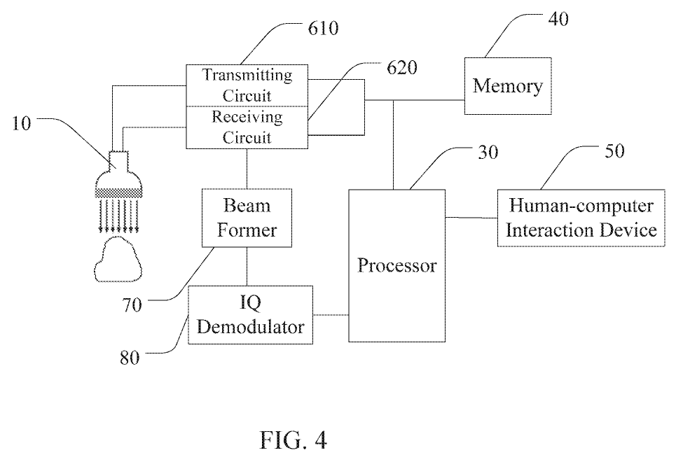

Referring to FIG. 4, the ultrasound imaging device may include an ultrasound probe 10, a transmitting circuit 610, a receiving circuit 620, a processor 30, a human-computer interaction device 50 and a memory 40.

The ultrasound probe 10 may be a catheter probe suitable for intracardiac ultrasound imaging, which is capable of being inserted into the heart chamber. The ultrasound probe 10 may include a transducer (not shown) including multiple array elements arranged in an array, such as in a two-dimensional array. The array elements are configured to transmit ultrasound waves according to excitation electrical signals, or to convert received ultrasound waves into electrical signals. Therefore, each array element may be configured to realize the mutual conversion between electrical pulse signals and ultrasound waves, thereby enabling the transmitting of ultrasound waves to the biological tissue at the site being scanned and the receiving of the echoes of ultrasound waves reflected back by the tissue. The ultrasound probe 10 may include array element groups. The array element group may include multiple array elements. Multiple transmitting aperture groups may be formed by the array element group. Each transmitting aperture group may include at least two transmitting apertures, and each transmitting aperture may include several array elements. One transmitting aperture may transmit the ultrasound beam towards the area being scanned along at least one scan line.

The transmitting circuit 610 may be configured to control the ultrasound probe 10 to transmit ultrasound waves. For example, under the control of the processor 30, the transmitting circuit 610 may excite the ultrasound probe 10 to transmit ultrasound waves toward the area being scanned.

The receiving circuit 620 may be configured to control the ultrasound probe 10 to receive the echoes of the ultrasound waves. For example, the receiving circuit 620 may receive, via the ultrasound probe 10, the ultrasound echoes returning from the area being scanned to obtain ultrasound echo signals, and may also process the ultrasound echo signals. The receiving circuit 620 may include one or more amplifiers, analog-to-digital converters (ADCs), etc.

The memory 40 may be configured to store various types of data.

The ultrasound imaging device may further include a beam former 70 and an IQ demodulator 80.

The beam former 70 may be connected to the receiving circuit 620, and be configured to perform beamforming processing on the echo signals, such as delay and weighted summation. Because the distances from the ultrasound receiving points in the tissue being examined to the receiving array elements are different, the channel data of the same receiving point outputted by different receiving array elements has differences in time delay. Therefore, delay processing may be performed to align the phases, and the weighted summation of the different channel data of the same receiving point may then be performed to obtain the beamformed ultrasound data. The ultrasound data outputted by the beam former 70 may be called radio frequency (RF) data. The beam former 70 may output the RF data to the IQ demodulator 80. In some embodiments, the beam former 70 may also output the RF data to the memory 40 for caching or storage, or directly output the RF data to the processor 30 for image processing.

The beam former 70 may perform the functions above using hardware, firmware or software. The beam former 70 may be integrated into the processor 30 or be arranged separately, which will not be limited by the present disclosure.

The IQ demodulator 80 may remove the signal carrier through IQ demodulation, extract the tissue structure information contained in the signal, and remove the noise by filtering. The acquired signals may be called the baseband signal (IQ data pair). The IQ demodulator 80 may output the IQ data pair to the processor 30 for image processing. In some embodiments, the IQ demodulator 80 may also output the IQ data pair to the memory 40 for caching or storage, so that the processor 30 can read the data from the memory 40 for subsequent image processing.

The IQ demodulator 80 may perform the functions above by hardware, firmware, or software. Similarly, the IQ demodulator 80 may be integrated into the processor 30 or be arranged separately, which will not be limited by the present disclosure.

The processor 30 may be implemented as a central control circuit (CPU), one or more microprocessors, a graphics processing unit (GPU) or any other electronic component that can process the inputted data according to specific logic instructions. The processor 30 can control the peripheral electronic components based on inputted instructions or predetermined instructions, or perform data reading and/or saving to the memory 40. The processor 30 can also process the inputted data by executing programs in the memory 40, such as performing one or more processing operations on the acquired ultrasound data according to one or more operating modes. These processing operations include, but are not limited to, adjusting or limiting the form of the ultrasound waves transmitted by the ultrasound probe 10, generating various image frames for display on the display of the human-machine interface device 50, adjusting or limiting the content and form displayed on the display, or adjusting one or more image display settings on the display (e.g., ultrasound images, interface components, location of region of interest, etc.).

Upon receiving the echo signals, the acquired ultrasound data may be processed in real- time by the processor 30 during the scanning, or be temporarily stored in the memory 40 and processed in a near-real-time manner during online or offline operation.

In an embodiment, the processor 30 may control the operations of the transmitting circuit 610 and the receiving circuit 620, such as controlling the transmitting circuit 610 and the receiving circuit 620 to operate alternately or simultaneously. The processor 30 may also determine a suitable operating mode according to the user's selection or program settings, such as a B-image mode, a C-image mode or a D-image mode (Doppler mode), etc., form a transmission sequence corresponding to the current operating mode, and send the transmission sequence to the transmitting circuit 610 such that the transmitting circuit 610 can use the appropriate transmission sequence to control the ultrasound probe 10 to transmit ultrasound waves.

The processor 30 may also be configured to process the ultrasound data to generate a grayscale image reflecting the signal intensity variations within the scanning range. This grayscale image reflects the internal anatomical structure of the tissue and is called a B-mode image. The processor 30 can output the B-mode image to the display of the human-computer interaction device 50 for display.

Under the user's operation and the control of the processor 30, various functions of the ultrasound imaging device can be realized.

Taking intracardiac echocardiography as an example, during the imaging process, the user can insert the ultrasound probe 10 from a peripheral blood vessel into the heart chamber. The ultrasound images can be acquired via the ultrasound probe 10, so as to realize the imaging of the heart and its adjacent tissues and/or the hemodynamic measurement.

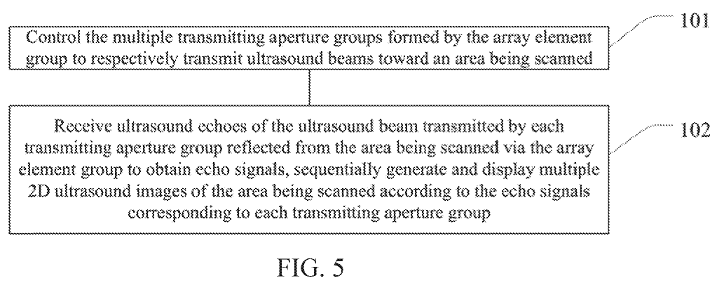

Referring to FIG. 5, the present disclosure provides an ultrasound imaging method, which may be controlled by the processor 30 and include the following steps.

In step 101, the processor 30 may control the multiple transmitting aperture groups formed by the array elements of the ultrasound probe 10 to respectively transmit ultrasound beams to the area being scanned. All the array elements in the ultrasound probe 10 may form one or more array element group. The array element group may form multiple transmitting aperture groups, each transmitting aperture group may include at least two transmitting apertures, and each transmitting aperture may be formed by several array elements in the array element group.

Before this step, the doctor may insert the ultrasound probe 10 to examine the heart chambers and identify the area being scanned. Specifically, the ultrasound probe 10 may be inserted into the heart chambers through the peripheral blood vessels to perform the ultrasound scan to obtain the ultrasound images, allowing the doctor to locate the area being scanned. That is, after completing the above preparations, the doctor may place the ultrasound probe 10 on the area being scanned and operate the ultrasound imaging device to issue the instruction for ultrasound scanning. In response to the instruction, the processor 30 may control the transmitting apertures of the multiple transmitting aperture groups formed by the array element group of the ultrasound probe 10 via the transmitting circuit 610 to transmit the ultrasound beams to the area being scanned.

In one embodiment, the transmitting aperture groups and the suitable positions of the transmitting apertures in the transmitting aperture groups may be determined by user selection or program settings. Based on the position of the transmitting aperture, the transmitting array elements corresponding to each transmitting aperture may be determined. The transmitting aperture may be formed by the corresponding transmitting array elements, and each transmitting aperture may be controlled to transmit the ultrasound beams along at least one scan line.

Several ways may be used to control the multiple transmitting aperture groups to respectively transmit the ultrasound beams to the area being scanned.

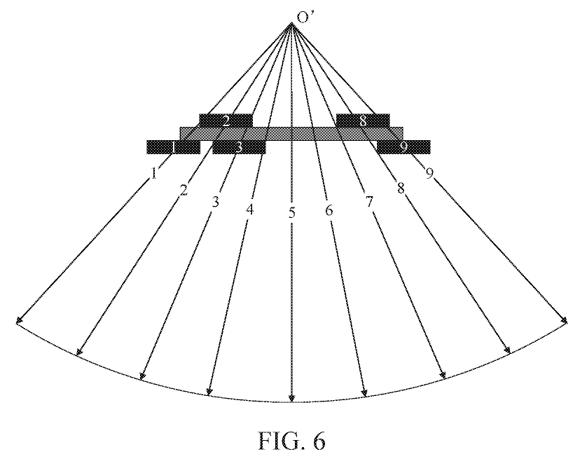

In the first way, for any one of the multiple transmitting aperture groups, the ultrasound beams are transmitted to the area being scanned by each transmitting aperture in the transmitting aperture group along at least one scan line corresponding to said transmitting aperture, and the multiple scan lines corresponding to the multiple transmitting apertures in the transmitting aperture group determine one scanning plane corresponding to said transmitting aperture group in space. Each scanning plane includes multiple scanning lines, the multiple scanning lines in a same scanning plane intersect at an endpoint in a direction opposite to a transmitting direction of the ultrasound beams, each scanning plane has a corresponding endpoint, and the endpoint is located on a side of the of the array element group opposite to the area being scanned. As shown in FIG. 6, scan lines 1, 2, ..., 9, which determine a scanning plane, are formed by the ultrasound beams transmitted by the transmitting apertures 1, 2, ..., 9, respectively, where the transmitting apertures 1, 2, ..., 9 belong to the same transmitting aperture group. Each transmitting aperture corresponds to one scan line, and these scan lines intersect at a virtual endpoint O' on one side of the ultrasound probe opposite to the area being scanned.

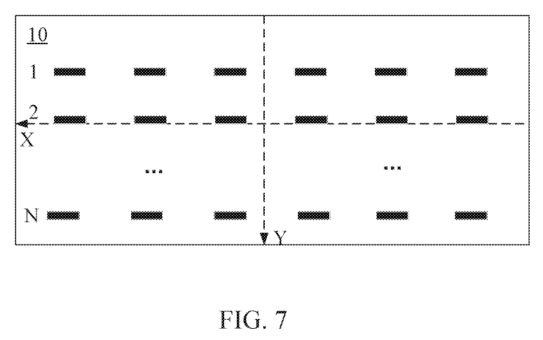

In some embodiments, the array element group may include N rows and M columns of transmitting apertures. At least two transmitting apertures in each row may form one transmitting aperture group. Each transmitting aperture group may correspond to one scanning plane. Therefore, N scanning planes may be formed. For example, as shown in FIG. 7, the at least two transmitting apertures in one row forming one transmitting aperture group may be the at least two transmitting apertures arranged along the X direction (horizontal direction) of the ultrasound probe 10.

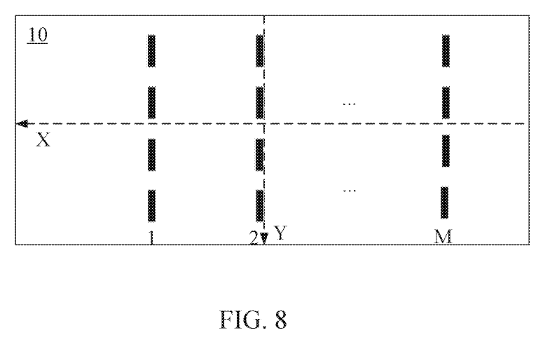

In some embodiments, the array element group may include N rows and M columns of transmitting apertures. At least two transmitting apertures in each column may form one transmitting aperture group. Each transmitting aperture group may correspond to one scanning plane. Therefore, M scanning planes may be formed. For example, as shown in FIG. 8, the at least two transmitting apertures in one column forming one transmitting aperture group may be the at least two transmitting apertures arranged along the Y direction (vertical direction) of the array element group.

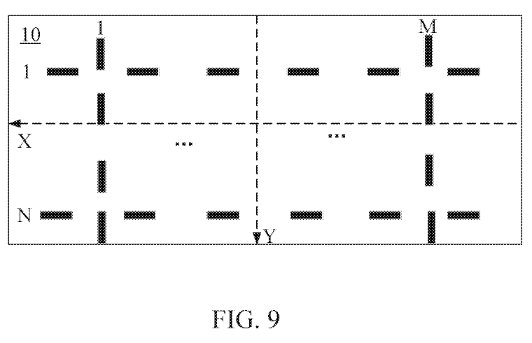

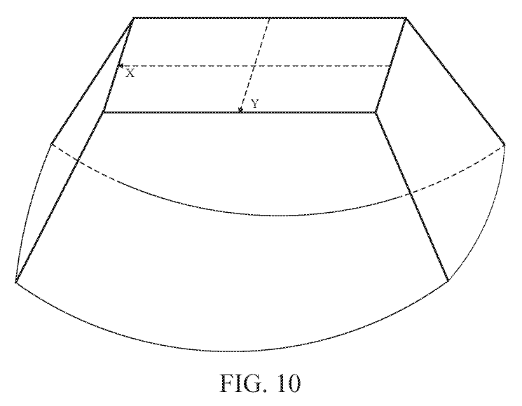

In some embodiments, the array element group may also include N rows and M columns of transmitting apertures. At least two transmitting apertures in each row may form one transmitting aperture group. Each transmitting aperture group may correspond to one scanning plane. Therefore, N scanning planes may be formed. At least two transmitting apertures in each column may form one transmitting aperture group. Each transmitting aperture group may correspond to one scanning plane. Therefore, M scanning planes may be formed. Thus, the N rows and M columns of transmitting apertures may form M+N transmitting aperture groups, that is, may determine M+N scanning planes. In one embodiment, the arrangement direction of the row of transmitting apertures in the N transmitting aperture groups may be perpendicular to the arrangement direction of the column of transmitting apertures in the M transmitting aperture groups. For example, as shown in FIG. 9, each row of transmitting apertures in the N transmitting aperture groups may be arranged along the X direction, and each column of transmitting apertures in the M transmitting aperture groups may be arranged along the Y direction, such that the resulting M+N scanning planes may form a three-dimensional scanning volume as shown in FIG. 10.

Furthermore, the multiple scanning planes determined by the multiple scanning lines corresponding to the at least two transmitting apertures in any one of the multiple transmitting aperture groups formed by the array element group may have various distribution patterns. Examples are given below.

In some embodiments, the endpoints corresponding to at least two of the multiple scanning planes may coincide. Taking a first scanning plane and a second scanning plane in the multiple scanning planes as examples, the endpoints corresponding to the first and second scanning planes coincide. In one embodiment, the arrangement direction of the transmitting array elements corresponding to the multiple scan lines in the first scanning plane may be parallel to the arrangement direction of the transmitting array elements corresponding to multiple scan lines in the second scanning plane. In other embodiments, the arrangement direction of the transmitting array elements corresponding to the multiple scan lines in the first scanning plane may be perpendicular to the arrangement direction of the transmitting array elements corresponding to multiple scan lines in the second scanning plane.

In some embodiments, the endpoints corresponding to at least two of the multiple scanning planes and the center of the array element group may be located on the same straight line. Taking a third scanning plane and a fourth scanning plane in the multiple scanning planes as examples, the endpoint corresponding to the third scanning plane and the endpoint corresponding to the fourth scanning plane may be located on a straight line passing through the center of the array element group in the vertical direction. In one embodiment, the arrangement direction of the transmitting array elements corresponding to the multiple scan lines in the third scanning plane may be parallel to the arrangement direction of the transmitting array elements corresponding to the multiple scan lines in the fourth scanning plane. In other embodiments, the arrangement direction of the transmitting array elements corresponding to the multiple scan lines in the third scanning plane may be perpendicular to the arrangement direction of the transmitting array elements corresponding to the multiple scan lines in the fourth scanning plane.

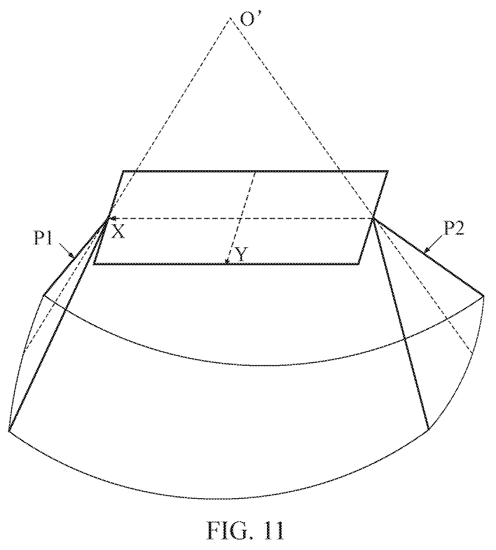

In the second way, the array element group of the ultrasound probe 10 may form multiple transmitting aperture groups, each transmitting aperture group may include at least two transmitting apertures, and each transmitting aperture may include several array elements. The multiple transmitting apertures may be arranged along a first direction. For any one of the multiple transmitting aperture groups, the multiple scan lines corresponding to the multiple transmitting apertures in the transmitting aperture group may determine one scanning plane corresponding to said transmitting aperture group in space. For the scanning planes corresponding to any two of the multiple transmitting aperture groups, the centerline of the scanning plane corresponding to one transmitting aperture group intersects the centerline of the scanning plane corresponding to the other transmitting aperture group at an endpoint in the direction opposite to the transmitting direction of the ultrasound beam, and the endpoint is located at one side of the array element group opposite to the area being scanned. For example, as shown in FIG. 11, the first direction is the X-axis direction, and the multiple transmitting aperture groups are arranged along the X-axis of the array element group. For any two scanning planes, such as the scanning plane P1 and the scanning plane P2, the centerline of the scanning plane P1 and the centerline of the scanning plane P2 intersect at an endpoint O' in the direction opposite to the transmitting direction of the ultrasound beams.

In some embodiments, the scanning planes above may be parallel to a second direction that is perpendicular to the first direction. For example, when the first direction is the X-axis direction, the second direction may be the Y-axis direction.

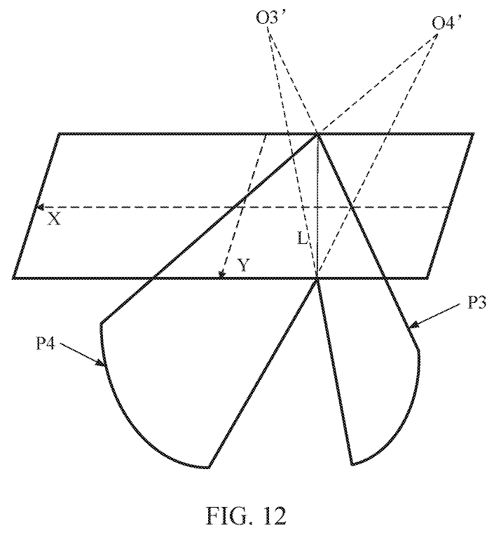

In the third way, the array element group of the ultrasound probe 10 may form multiple transmitting aperture groups, each transmitting aperture group may include at least two transmitting apertures, and each transmitting aperture may include several array elements. For any one of the multiple transmitting aperture groups, the multiple scan lines corresponding to the multiple transmitting apertures in the transmitting aperture group may determine one scanning plane corresponding to said transmitting aperture group in space. Each scanning plane includes multiple scanning lines. The multiple scanning lines in the same scanning plane intersect at an endpoint in the direction opposite to the transmitting direction of the ultrasound beam. Each scanning plane has a corresponding endpoint, and the endpoint is located on one side of the array element group opposite to the area being scanned. The scanning planes corresponding to the multiple transmitting aperture groups may intersect the array element group at the same straight line, and the straight line may be located on the array element group. For example, as shown in FIG. 12, for any two scanning planes among the multiple scanning planes, such as the scanning plane P3 and the scanning plane P4, they intersect the array element group at a straight line L. The scanning lines in the scanning plane P3 intersect at the endpoint 03', and the scanning lines in the scanning plane P4 intersect at the endpoint 04'.

Furthermore, the embodiments of the present disclosure also provide some examples of scanning planes formed by different arrangements of the transmitting array elements.

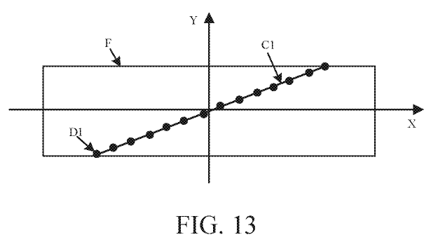

In some embodiments, as shown in FIG. 13, the projection of the array element group of the ultrasound probe 10 in the vertical direction is rectangle F. The multiple transmitting apertures formed by multiple array elements D1 of the array element group correspond to a scanning plane, and the projection of the scanning plane in the vertical direction is the projection line C1. The projection line C1 passes through the center of rectangle F.

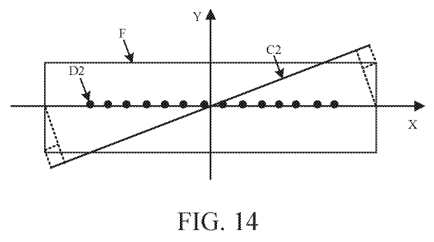

In some embodiments, as shown in FIG. 14, the projection of the array element group of the ultrasound probe 10 in the vertical direction is rectangle F. The multiple transmitting apertures formed by multiple array elements D1 of the array element group correspond to a scanning plane, and the projection of the scanning plane in the vertical direction is the projection line C2. The projection line C2 passes through the center of rectangle F.

It should be noted that the vertical direction may refer to the direction perpendicular to the plane on which the array elements are located. The projection lines (C1, C2) are the intersecting line segments formed by the intersection of the scanning plane with the array elements.

By calculation of the length of the projection line of the scanning plane in the vertical direction, the actual scanning range of the ultrasound probe may be determined. As shown in FIG. 14, the length of the projection line C2 exceeds the actual range of the array element group. That is, the scanning with the scanning plane formed by the transmitting array elements shown in FIG. 14 can obtain a larger scanning range.

After the processor 30 control the multiple transmitting aperture groups formed by the array element group of the ultrasound probe 10 to respectively transmit the ultrasound beams to the area being scanned, the process may further include the following steps.

In step 102, the processor 30 may receive the echo signals of the ultrasound echoes of the ultrasound beams transmitted by each transmitting aperture group reflected from the area being scanned via the array element group, and sequentially generate multiple 2D ultrasound images of the area being scanned based on the echo signals corresponding to each transmitting aperture group and display the multiple 2D ultrasound images. In some embodiments, the multiple 2D ultrasound image may be displayed in real time. That is, each time one 2D ultrasound image is acquired, it is displayed.

The present disclosure has been described with reference to various exemplary embodiments. However, those skilled in the art will recognize that changes and modifications can be made to the exemplary embodiments without departing from the scope of the present disclosure. For example, various steps and components configure to perform the steps may be implemented in different ways depending on the specific application or considering any number of cost functions associated with the operation of the system (e.g., one or more steps may be deleted, modified, or combined with other steps).

Furthermore, as those skilled in the art will understand, the principles of the present disclosure may be implemented in a computer program product on a computer-readable storage medium pre-loaded with computer-readable program code. Any tangible, non- transitory computer-readable storage medium may be used, including magnetic storage devices (hard disks, floppy disks, etc.), optical storage devices (CD-ROMs, DVDs, Blu-ray discs, etc.), flash memory, and/or the like. The computer program instructions may be loaded onto a general-purpose computer, a special-purpose computer, or other programmable data processing apparatus to form a machine, such that the instructions that are execute on a computer or other programmable data processing apparatus can form the device for implementing a specified function. The computer program instructions may also be stored in a computer-readable storage medium that can instruct a computer or other programmable data processing apparatus to operate in a particular manner, such that the instructions stored in the computer-readable storage medium can form an article of manufacture, including the device for implementing the specified function. The computer program instructions may also be loaded onto a computer or other programmable data processing apparatus to perform a series of operational steps on the computer or the programmable apparatus to produce a computer-implemented process, such that the instructions that are execute on the computer or other programmable apparatus can provide steps for implementing the specified function.

Although the principles of the present disclosure have been illustrated in various embodiments, numerous modifications to the structure, arrangement, proportions, elements, materials, and components that are particularly suitable for specific environmental and operational requirements may be used without departing from the principles and scope of the present disclosure. The modifications above, and other alterations or changes, will be included within the scope of the present disclosure.

The foregoing specific descriptions have been provided with reference to various embodiments. However, those skilled in the art will recognize that various modifications and changes can be made without departing from the scope of the present disclosure. Therefore, the present disclosure are illustrative rather than restrictive, and all such modifications are to be included within its scope. Similarly, the advantages and solutions to problems of various embodiments have been described above. However, benefits, advantages, solutions to problems, and any elements that produce these or solutions making them more explicit, should not be construed as critical, essential, or necessary. The term "comprising" and any other variations thereof as used herein are non-exclusive inclusion, meaning that the process, method, article, or apparatus that includes a list of elements include not only those elements but also other elements not expressly listed or not part of the process, method, system, article, or apparatus. Furthermore, the term "coupled" and any other variations thereof as used herein refer to physical connections, electrical connections, magnetic connections, optical connections, communication connections, functional connections, and/or any other connections.

Those skilled in the art will recognize that many changes can be made to the details of the embodiments above without departing from the basic principles of the present disclosure. Therefore, the scope of the present disclosure should be determined according to the claims.

1. An ultrasound imaging device, comprising:an ultrasound probe, wherein, the ultrasound probe comprises an array element group comprising multiple array elements, the array element group forms multiple transmitting aperture groups, each transmitting aperture group comprises at least two transmitting apertures, each transmitting aperture comprises multiple array elements, and each transmitting aperture transmits ultrasound beams along at least one scan line corresponding to said transmitting aperture; anda processor configured to:control the multiple transmitting aperture groups formed by the array element group to respectively transmit ultrasound beams toward an area being scanned, wherein:for any one of the multiple transmitting aperture groups, the ultrasound beam is transmitted to the area being scanned by each transmitting aperture in the transmitting aperture group along at least one scan line corresponding to said transmitting aperture, and the multiple scan lines corresponding to the multiple transmitting apertures in the transmitting aperture group determine one scanning plane corresponding to said transmitting aperture group in space, wherein, each scanning plane comprises multiple scanning lines, the multiple scanning lines in a same scanning plane intersect at an endpoint in a direction opposite to a transmitting direction of the ultrasound beams, each scanning plane has a corresponding endpoint, and the endpoint is located on a side of the of the array element group opposite to the area being scanned;receive ultrasound echoes of the ultrasound beam transmitted by each transmitting aperture group reflected from the area being scanned via the array element group to obtain echo signals;generate multiple 2D ultrasound images of the area being scanned according to the echo signals corresponding to each transmitting aperture group; anddisplay the multiple 2D ultrasound images.

2. The ultrasound imaging device of claim 1, wherein the array element group comprises N rows and M columns of transmitting apertures, wherein at least two transmitting apertures in each row form one transmitting aperture group; and/or at least two transmitting apertures in each column form one transmitting aperture group.

3. The ultrasound imaging device of claim 1, wherein, the multiple scanning planes corresponding to the multiple transmitting aperture groups comprise a first scanning plane and a second scanning plane, and the endpoint corresponding to the first scanning plane coincide with the endpoint corresponding to the second scanning plane.

4. The ultrasound imaging device of claim 3, wherein an arrangement direction of transmitting array elements corresponding to the multiple scan lines in the first scanning plane is parallel or perpendicular to an arrangement direction of transmitting array elements corresponding to the multiple scan lines in the second scanning plane.

5. The ultrasound imaging device of claim 1, wherein, the scanning planes corresponding to the multiple transmitting apertures comprise a third scanning plane and a fourth scanning plane, and the endpoint corresponding to the third scanning plane, the endpoint corresponding to the fourth scanning plane and a center of the array element group are located on a same straight line.

6. The ultrasound imaging device of claim 5, wherein the endpoint corresponding to the third scanning plane and the endpoint corresponding to the fourth scanning plane are located on the straight line passing through the center of the array element group in a vertical direction.

7. The ultrasound imaging device of claim 5, wherein an arrangement direction of transmitting array elements corresponding to the multiple scanning lines in the third scanning plane is parallel or perpendicular to an arrangement direction of transmitting array elements corresponding to the multiple scanning lines in the fourth scanning plane.

8. The ultrasound imaging device of claim 1, wherein, a projection of the array element group in a vertical direction is a rectangle, and a projection line of the scanning plane in a vertical direction passes through a center of the rectangle.

9. The ultrasound imaging device of claim 1, wherein, the multiple transmitting apertures are arranged along a first direction, and the scanning planes corresponding to the transmitting aperture groups are parallel to a second direction perpendicular to the first direction.

10. The ultrasound imaging device of claim 1, wherein, for the scanning planes corresponding to any two transmitting aperture groups in the multiple transmitting aperture groups, a centerline of the scanning plane corresponding to one transmitting aperture group and a centerline of the scanning plane corresponding to the other transmitting aperture group intersect at an endpoint in a direction opposite to a transmitting direction of the ultrasound beams and the endpoint is located on a side of the area being scanned opposite to the array element group.

11. The ultrasound imaging device of claim 1, wherein, the scanning planes corresponding to the
multiple transmitting aperture groups intersect the array element group at a same straight line located on the array element group.

12. An ultrasound imaging method applied to an ultrasound imaging device, wherein, the ultrasound imaging device comprises an ultrasound probe comprising an array element group, the array element group comprises multiple array elements, the array element group forms multiple transmitting aperture groups, each transmitting aperture group comprises at least two transmitting apertures, each transmitting aperture comprises several array elements, one transmitting aperture transmits ultrasound beams along at least one scanning line corresponding to said one transmitting aperture, and the method comprises:control the multiple transmitting aperture groups formed by the array element group to respectively transmit the ultrasound beams to an area being scanned, wherein for any one of the multiple transmitting aperture groups, the ultrasound beam is transmitted toward the area being scanned by each transmitting aperture in the transmitting aperture group along at least one scan line corresponding to said transmitting aperture, and the multiple scan lines corresponding to the multiple transmitting apertures in the transmitting aperture group determine one scanning plane corresponding to said transmitting aperture group in space, wherein, each scanning plane comprises multiple scanning lines, the multiple scanning lines in a same scanning plane intersect at an endpoint in a direction opposite to a transmitting direction of the ultrasound beams, each scanning plane has a corresponding endpoint, and the endpoint is located on a side of the of the array element group opposite to the area being scanned;receive ultrasound echoes of the ultrasound beam transmitted by each transmitting aperture group reflected from the area being scanned via the array element group to obtain echo signals;generate multiple 2D ultrasound images of the area being scanned according to the echo signals corresponding to each transmitting aperture group; anddisplay the multiple 2D ultrasound images.

13. The method of claim 12, wherein the array element group comprises N rows and M columns of transmitting apertures, wherein at least two transmitting apertures in each row form one transmitting aperture group; and/or,at least two transmitting apertures in each column form one transmitting aperture group.

14. The method of claim 12, wherein, the scanning planes corresponding to the multiple transmitting aperture groups comprises a first scanning plane and a second scanning plane, and the endpoint corresponding to the first scanning plane coincide with the endpoint corresponding to the
second scanning plane.

15. The method of claim 14, wherein an arrangement direction of transmitting array elements corresponding to the multiple scan lines in the first scanning plane is parallel or perpendicular to an arrangement direction of transmitting array elements corresponding to the multiple scan lines in the second scanning plane.

16. The method of claim 12, wherein, the scanning planes corresponding to the multiple transmitting aperture groups comprises a third scanning plane and a fourth scanning plane, and the endpoint corresponding to the third scanning plane, the endpoint corresponding to the fourth scanning plane and a center of the array element group are located on a same straight line.

17. The method of claim 16, wherein the endpoint corresponding to the third scanning plane and the endpoint corresponding to the fourth scanning plane are located on the straight line passing through the center of the array element group in a vertical direction.

18. The method of claim 16, wherein an arrangement direction of transmitting array elements corresponding to the multiple scanning lines in the third scanning plane is parallel or perpendicular to an arrangement direction of transmitting array elements corresponding to the multiple scanning lines in the fourth scanning plane.

19. The method of claim 12, wherein, a projection of the array element group in a vertical direction is a rectangle, and a projection line of the scanning plane in a vertical direction passes through a center of the rectangle.

20. A computer-readable storage medium storing a program, wherein the program is executed by a processor to:control multiple transmitting aperture groups formed by an array element group of an ultrasound probe of an ultrasound imaging device to respectively transmit ultrasound beams to an area being scanned, wherein for any one of the multiple transmitting aperture groups, the ultrasound beam is transmitted toward the area being scanned by each transmitting aperture in the transmitting aperture group along at least one scan line corresponding to said transmitting aperture, and the multiple scan lines corresponding to the multiple transmitting apertures in the transmitting aperture group determine one scanning plane corresponding to said transmitting aperture group in space, wherein, each
scanning plane comprises multiple scanning lines, the multiple scanning lines in a same scanning plane intersect at an endpoint in a direction opposite to a transmitting direction of the ultrasound beams, each scanning plane has a corresponding endpoint, and the endpoint is located on a side of the of the array element group opposite to the area being scanned;receive ultrasound echoes of the ultrasound beam transmitted by each transmitting aperture group reflected from the area being scanned via the array element group to obtain echo signals;generate multiple 2D ultrasound images of the area being scanned according to the echo signals corresponding to each transmitting aperture group; anddisplay the multiple 2D ultrasound images.
